(19) United States
(12) Patent Application Publication
(10) Pub. No.: US 2021/0098089 A1
(43) Pub. Date: Apr. 1, 2021

(54) METHOD AND SYSTEM FOR PROVIDING PATIENT-CONDITION-CHECK-DISPLAY

(71) Applicant: Visual Terminology Inc., Busan (KR)

(72) Inventor: Byung Kwan Choi, Busan (KR)

(21) Appl. No.: 16/585,129

(22) Filed: Sep. 27, 2019

(51) Int. Cl. G16H 10/60; G16H 30/40; G16H 50/20

(52) U.S. Cl. CPC G16H 10/60; G16H 50/30; G16H 50/20; G16H 30/40

(57) ABSTRACT

Disclosed is a method and system for providing a patient-condition-check-display. The method includes identifying a disease occurring in a patient from a medical record of the patient, and visualizing, in a body image, a disease image representing the disease based on an occurrence time of the disease and providing the patient-condition-check-display including the disease image visualized in the body image.

### BACKGROUND

### 1. Field

One or more example embodiments relate to a method and system for providing a patient-condition-check-display to enable an easy search for a medical history and a current condition of a patient based on an image visualized based on a lapse of time, and more particularly, to a method and system for providing a patient-condition-check-display to display and visualize a lesion on a body map based on medical history information of a patient, track a development of the lesion based on a lapse of time, and allow a user to readily verify and search for progression of the disease at a certain time through a graphical user interface (GUI) controller, for example, a scroll bar.

### 2. Description of Related Art

In general, doctors practicing in a medical field examine patients using medical tools such as stethoscopes, verify a result of the examination, view patient medical history charts, directly ask patients about their past and current conditions to verify their medical histories and current conditions, and make a diagnosis and prescribe medicine accordingly.

However, patients may not exactly remember diseases they suffered or surgeries they underwent, and may not inform doctors about the surgeries or medicine they took or are taking unless they are asked for such information by the doctors even though this information may be closely related to diseases they are experiencing currently. In addition, when a medical chart includes too many records related to diseases and symptoms a patient had or is still having, a doctor may overlook an important record about a past disease and a medical treatment the patient experienced.

Further, an existing medical chart, for example, an electronic medical record (EMR), is provided in a form of free-text and difficult for a computer to read. Thus, when other medical practitioners or doctors examine the same patient, they may not be able to accurately diagnose a current condition of the patient within a short period of time even though they refer to the medical record.

Thus, there is a desire for technology for rapidly and accurately determining the progression of diseases already occurring in a patient, and a relationship between the diseases.

### SUMMARY

An aspect provides a method and system that may provide, to an electronic medical record (EMR) display run in a terminal of a medical practitioner, a patient-condition-check-display that visualizes, in a body image, an image of a disease developed in a patient based on a lapse of time, and displays the visualized image. The method and system may thus allow the medial practitioner to readily verify a medical history and a current medical condition of a patient through an image in lieu of a complex text.

In addition, the method and system may allow the medical practitioner to readily search for and retrieve progression or development of a disease at a specific point in time simply by using a graphical user interface (GUI) controller.

Further, the method and system may allow the medical practitioner to verify, all at once, occurrence and procession of a disease and a process of a medical treatment for the disease, using an image based on a lapse of time, simply through an operation of the GUI controller.

According to an example embodiment, there is provided a method of providing a patient-condition-check-display, the method including identifying a disease occurring in a patient from a medical record of the patient, and visualizing, in a body image, a disease image representing the disease based on an occurrence time of the disease and providing the patient-condition-check-display including the disease image visualized in the body image.

According to another example embodiment, there is provided a method of providing a patient-condition-check-display, the method including selecting, as a region of interest (ROI), a region dragged or clicked in a body image on the patient-condition-check-display, identifying a disease occurring in a body tissue located in the ROI from a medical record of a patient, and visualizing a disease image representing the disease in the body image based on an occurrence time of the disease and providing the patient-condition-check-display including the disease image visualized in the body image. When a time to be displayed on the patient-condition-check-display changes by an operation of a first GUI controller, the method may include changing a color, a level of brightness, and a level of transparency of the disease image based on medical information including data of a treatment progress of the disease, and visualizing a state of the treatment progress of the disease.

According to still another example embodiment, there is provided a system for providing a patient-condition-check-display, the system including an identifier configured to identify a disease occurring in a patient from a medical record of the patient, in response to a request for the patient-condition-check-display, and a processor configured to visualize, in a body image, a disease image representing the disease based on an occurrence time of the disease and provide the patient-condition-check-display including the disease image visualized in the body image.

According to yet another example embodiment, there is provided a system for providing a patient-condition-check-display, the system including an identifier configured to select, as an ROI, a region dragged or clicked in a body image on the patient-condition-check-display, and identify a disease occurring in a body tissue located in the ROI from a medical record of a patient, and a processor configured to visualize a disease image representing the disease in the body image based on an occurrence time of the disease and provide the patient-condition-check-display including the disease image visualized in the body image. When a time to be displayed on the patient-condition-check-display changes by an operation of a first GUI controller, the processor may change a color, a level of brightness, and a level of transparency of the disease image based on medical information including data of a treatment progress of the disease, and visualize a state of the treatment progress of the disease.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the present disclosure will become apparent and more readily appreciated from the following description of example embodiments, taken in conjunction with the accompanying drawings of which:

FIG. 1 is a diagram illustrating an example of a system for providing a patient-condition-check-display according to an example embodiment;

FIG. 2 is a diagram illustrating an example of how a system for providing a patient-condition-check-display outputs a prescription registration display to a terminal of a doctor in connection with the patient-condition-check-display according to an example embodiment;

FIG. 3 is a diagram illustrating an example of a patient-condition-check-display provided by a system for providing a patient-condition-check-display according to an example embodiment;

FIGS. 4A through 4C are diagrams illustrating an example of visualizing occurrence and progression of a disease on a patient-condition-check-display provided by a system for providing a patient-condition-check-display according to an example embodiment;

FIGS. 5A and 5B are diagrams illustrating an example of visualizing occurrence and progression of a disease through speech bubbles on a patient-condition-check-display provided by a system for providing a patient-condition-check-display according to an example embodiment;

FIGS. 6A and 6B are diagrams illustrating an example of searching for a disease occurring at a certain time from a patient-condition-check-display provided by a system for providing a patient-condition-check-display according to an example embodiment; and

FIG. 7 is a flowchart illustrating an example of a method of providing a patient-condition-check-display according to an example embodiment.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known in the art may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

The terminology used herein is for the purpose of describing particular embodiments only and is not intended to be limiting. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, operations, elements, and/or components, but do not preclude the presence or addition of one or more other features, integers, operations, elements, components, and/or groups thereof.

Terms such as first, second, A, B, (a), (b), and the like may be used herein to describe components. Each of these terminologies is not used to define an essence, order, or sequence of a corresponding component but used merely to distinguish the corresponding component from other component(s). For example, a first component may be referred to as a second component, and similarly the second component may also be referred to as the first component.

Unless otherwise defined, all terms, including technical and scientific terms, used herein have the same meaning as commonly understood by one of ordinary skill in the art to which this disclosure pertains based on an understanding of the present disclosure. Terms, such as those defined in commonly used dictionaries, are to be interpreted as having a meaning that is consistent with their meaning in the context of the relevant art and the present disclosure, and are not to be interpreted in an idealized or overly formal sense unless expressly so defined herein.

Hereinafter, some example embodiments will be described in detail with reference to the accompanying drawings. Regarding the reference numerals assigned to the elements in the drawings, it should be noted that the same elements will be designated by the same reference numerals, wherever possible, even though they are shown in different drawings.

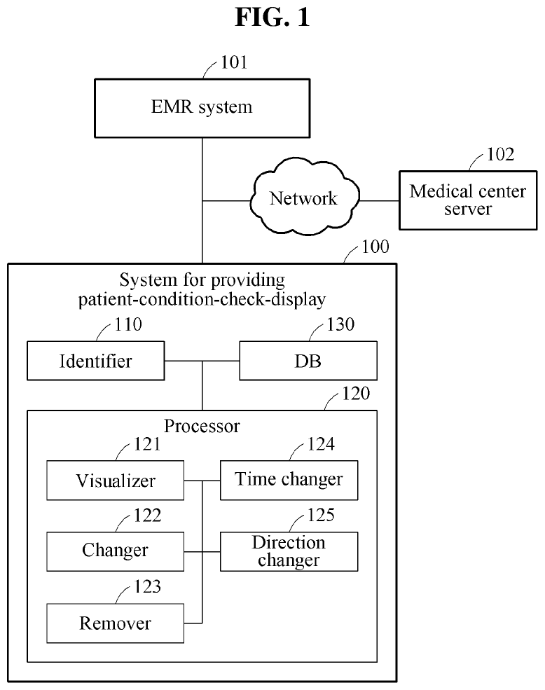

FIG. 1 is a diagram illustrating an example of a system for providing a patient-condition-check-display according to an example embodiment. Hereinafter, a system for providing a patient-condition-check-display will be simply referred to as a patient-condition-check-display providing system. A patient-condition-check-display may be a patient disease state display screen that displays a state of a disease experienced by a patient.

Referring to FIG. 1, a patient-condition-check-display providing system 100 includes an identifier 110, a processor 120, and a database (DB) 130.

The patient-condition-check-display providing system 100 may configure a patient-condition-check-display by referring to a patient medical record in an electronic medical record (EMR) system 101.

The patient-condition-check-display providing system 100 may display, via a medical center server 102, the patient-condition-check-display on a portion of an EMR display operating in a terminal of a doctor who treats a patient, or output the patient-condition-check-display in replacement of the EMR display.

The patient-condition-check-display may be output in a form of, for example, at least one of a window, an icon, or a graphical user interface (GUI) of a program, and include a body image and a GUI controller.

In addition, the patient-condition-check-display may further include a clock, a calendar, a graph, a coordinate, a progress bar, and a time indicating portion to indicate a time or a lapse of time that is indicated by the GUI controller.

The GUI controller may be a portion used to recognize an operation or manipulation of a user performed on the patient-condition-check-display, and be configured to operate by an input of a combination of one or more of a mouse, gravity, vibration, a touch sensor, a microphone, and a camera. In addition, the patient-condition-check-display providing system 100 may display a lapse of time or a time point that is indicated by the GUI controller in a form of a length, an angle, a volume, a numeral, or a text, to inform a user of such information.

For example, the GUI controller may be embodied by a form of a scroll bar providing a display-switching (or changing) function, and also be embodied by a region or portion that recognizes at least one of movements of other various buttons (e.g., play button, back button, fast button, setting button, radio button, and the like), a checkbox, a dropdown menu, a slider, a progress bar, a touch, a click, a cursor, and the like.

The body image may be embodied as an image including at least one of an entire body of a human being, a portion of the body, or a cross section of the body (e.g., cross-sectional image of the body captured through computerized tomography [CT] or magnetic resonance imaging [MRI]). In addition, the body image may be a body image of a patient that is constructed by combining these medical images of the patient.

Thus, through an image, the doctor (or a medical practitioner) may simply search for a medical history and a current condition of the patient from the patient-condition-check-display that is output along with the EMR display, simply by an operation of the GUI controller. When the doctor accurately recognizes a condition of the patient or a disease state of the patient through the simple search, the doctor may prescribe medication or determine a type of treatment suitable for the patient, for example, hospitalization and a surgery.

The EMR system 101 may refer to a system that computerizes and stores medical information including at least one of personal information of a patient, a medical history of the patient, a medical record or medical treatment record of the patient, or a hospitalization and discharge record of the patient, and provides, to the medical center server 102, the medical information of the patient in a form of an electronic chart upon request from a medical center with which the patient agrees on the provision of information. A medical center used herein may encompass all types of institutions, organizations, hospitals, clinics, and the like that provide medical services. A medical practitioner used herein may indicate all types of people who provide legal or legitimate medical services to patients.

In the example of FIG. 1, in response to a request for a patient-condition-check-display, the identifier 110 may identify a disease occurring in a patient from a medical record of the patient.

The identifier 110 may recognize a request for the patient-condition-check-display, when an item associated with searching for a medical history of a patient, or an item associated with registering a prescription or an order is selected from the EMR display. The identifier 110 may also recognize a request when an order or a type of treatment such as hospitalization, surgery, examination, and the like is input, or one patient is selected from a list of patients or when a medical treatment is initiated thereby.

The identifier 110 may search for the medical record of the patient from the EMR system 101, and identify all diseases or disorders previously occurring in the patient based on a current time at which the request is made.

The identifier 110 may search from the EMR system 101 for all medical records of the patient including a medical record of the patient issued by a medical center that is currently visited by the patient, and medical records or medical examination records of the patient issued by other medical centers including medical examination centers that were previously visited by the patient.

For example, the identifier 110 may select and identify, from the medical record of the patient, a disease with a relatively high level of severity, for example, a thyroid disease, a heart disease, and a lung disease, excluding a disease with a low level of severity, for example, a common cold, a stomachache, and a headache.

In addition, the identifier 110 may select and identify, from the medical record of the patient, a certain disease (e.g., cerebral hemorrhage), diseases associated with each other (e.g., high blood pressure and a heart disease), a disease occurring in a region set as a region of interest (ROI) (e.g., lung), and a disease occurring at a certain time, for example, a time after a certain type of medicine or drug is administered to a patient at an emergency room.

In addition, when identifying a disease from the medical record of the patient, the identifier 110 may further identify data of a medical treatment process including, for example, an occurrence time of the disease, a full recovery time at which the disease is completely cured, an occurrence region at which the disease occurs in a body of the patient, a size and a shape of the disease, an administered medicine or drug and a surgery, hospitalization, an examination or checkup result, and a nursing record.

For example, the identifier 110 may identify, from the EMR system 101, diseases previously occurring in the patient by searching for the medical record of the patient from the EMR system 101, at a time that is not explicitly indicated or set or at an arbitrary time.

In addition, the identifier 110 may identify the diseases previously occurring in the patient by searching for the medical record of the patient from the EMR system 101 at a time when patient information is registered, a time when a request for outpatient care is received, or a time set for each day.

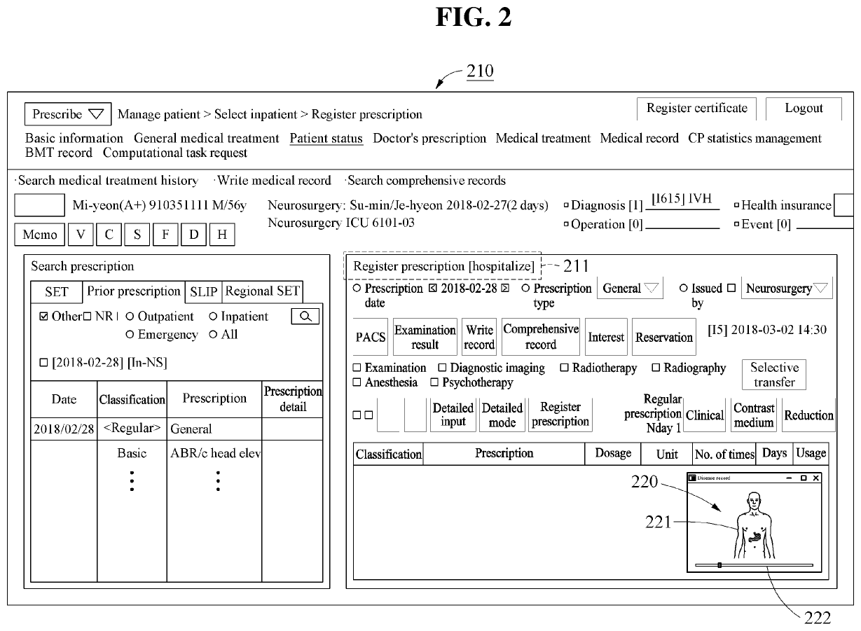

In addition, the identifier 110 may identify the diseases previously occurring in the patient by searching for the medical record of the patient from the EMR system 101 when an item associated with registering a prescription, for example, an item 211 illustrated in FIG. 2, is selected or pressed from the EMR display, for example, an EMR display 210 illustrated in FIG. 2, that is output to a terminal of the doctor who examines or provides a medical treatment to the patient.

The processor 120 may visualize, in a body image, a disease image representing a disease based on an occurrence time of the disease and provide the patient-condition-check-display including the disease image visualized in the body image.

That is, the processor 120 may sequentially visualize, in the body image, images representing diseases that change based on occurrence and progression of the diseases, and a treatment progress of the diseases.

In addition, the processor 120 may change a color of a location in the body image, for example, an organ, at which the disease occurs, or adjust a level of transparency of the location, and visualize a change in the organ based on the occurrence of the disease and the treatment progress of the disease.

In addition, the processor 120 may insert or visualize an icon or an arrow for each disease in the body image, and visualize the occurrence of the disease, and an occurrence location and a type of the disease.

In addition, the processor 120 may assign numerical numbers to diseases occurring in the patient from a previous time up to a current time based on respective occurrence times of the diseases, and sequentially visualize, in the body image, the diseases at respective locations at which the diseases occur based on the occurrence times of the diseases. Thus, the processor 120 may provide the doctor with guidance on the occurrence and the location of each of the diseases and a chronological sequence of the occurrence of the diseases.

In addition, when a GUI event occurs at a disease occurrence location in the body image, the processor 120 may display a speech bubble in association with the disease occurrence location, and visualize, in a form of text, information associated with a name of a disease, an occurrence time of the disease, a level of severity of the disease, and a current state of the disease through the speech bubble. The GUI event may be, for example, an input event occurring on the patient-condition-check-display through a user's operation, for example, a mouse click, a mouse-over, a mouse wheel, a mouse leaving operation, a GUI focusing operation, a keyboard input, a finger touch, a sound recognition operation, a body gesture or movement recognition operation, and the like.

For example, the processor 120 may output the speech bubble in association with a region in the body image that the user touches or a region to which a mouse cursor moves, and visualize, in a text form, detailed information associated with a disease occurring in the region.

For example, when a GUI event, for example, mouse focusing, clicking, and touching operations, occurs in the body image, the processor 120 may change the EMR display operating in the terminal of the doctor who requests the patient-condition-check-display. Thus, the processor 120 may provide a function of changing or switching the EMR display through the patient-condition-check-display, without providing an additional context menu.

Alternatively, the processor 120 may change the EMR display by providing the context menu on the patient-condition-check-display. In detail, when a user places a mouse on the body image of the patient-condition-check-display or clicks twice the mouse on the body image, the processor 120 may display the context menu including at least one of items including, for example, ‘display information,’ ‘prescribe,’ ‘search progression,’ and ‘record progression.’ When the item of ‘display information’ is selected from the context menu, the processor 120 may change an entirety or a portion of the EMR display output to the terminal of the doctor to a display indicating detailed information of a disease occurring.

Through such a display change or switch, the processor 120 may allow the doctor to verify, for example, an operation record, a blood test result, or a nursing record of the patent that is written or recorded based on a time at which the item of ‘display information’ is selected.

In addition, when the item of ‘prescribe’ is selected, the processor 120 may change an entirety or a portion of the EMR display to a display for writing or recording a prescription for the disease. When the item of ‘search progression’ is selected, the processor 120 may change an entirety or a portion of the EMR display to a display for displaying progression of the disease. When the item of ‘record progression’ is selected, the processor 120 may change an entirety or a portion of the EMR display to a display for writing or recording the progression of the disease.

Such an EMR display switching function may be performed by a shortcut key input. For example, when a scroll bar indicates a certain point in time and the patient receives a medical treatment for a corresponding disease, the doctor may search for a progression record by pressing a shortcut key ‘P,’ or search for a radiograph reading by pressing a shortcut key ‘R.’

In addition, when the doctor clicks an organ in the body image, the doctor may search for medical information predetermined for a corresponding disease, or request for a function of allowing him/her to write or record such information.

A disease image representing a disease used herein may be an image which resembles and represents an organ or a body tissue at which the disease occurs, or a lesion image of the disease, for example, a CT image, or an icon assigned to the disease. Here, an icon may be assigned to each disease.

A body image used herein may be an image including at least one of an entirety, a portion, or a cross section (e.g., cross-sectional image of a body captured through CT or MRI) of a body of a patient. Alternatively, the body image may be an image of the body of the patient that is constructed by combining all medical images of the patient.

A direction in which the body image is to be displayed and a size of the body image may change through an operation of a second GUI controller, for example, a setting button or a shortcut key included in the patient-condition-check-display. When the direction changes, an upper body or a lower body, a front side of the body, or a back side of the body may be displayed as the body image. Such change may facilitate stereoscopic visualization of a disease image representing a disease, and thereby facilitating observation of a lesion or the disease.

The processor 120 may sequentially visualize, in the body image, disease images of diseases identified from the medical record of the patient based on respective occurrence times of the diseases, and allow the doctor to readily verify the occurrence of a disease based on a time, and a change or progression of the disease based on a lapse of time.

In addition, the processor 120 may visualize, in the body image, all the disease images all together, and provide a disease map indicating all the disease the patient suffers previously, thereby allowing the doctor to verify the diseases all at once.

In addition, the processor 120 may classify, into a same group, diseases occurring at a similar time or at a similar location among the diseases identified from the medical record of the patient, and visualize the diseases in the same group in the body image, thereby allowing the doctor to verify a relationship or correlation between the diseases.

For example, referring to FIG. 2, the processor 120 may provide a patient-condition-check-display 220 including a body image 221 and a GUI controller 222 to an EMR display 210, and visualize a disease image (e.g., shape of stomach) representing an identified disease or disorder (e.g., stomach disorder) in a disease occurrence region (e.g., center in an upper human body) in the body image 221. The processor 120 may then change the disease image to be visualized in the body image 221 by applying a treatment state of the disease based on an operation of the GUI controller 222, for example, a movement of a scroll bar.

For another example, as a time, which indicates a point in time, changes in a direction receding from a reference point, the identifier 110 may verify a time newly selected by an operation of the GUI controller, and identify a first disease occurring at the verified time and a second disease occurring prior to the time and not completely cured yet.

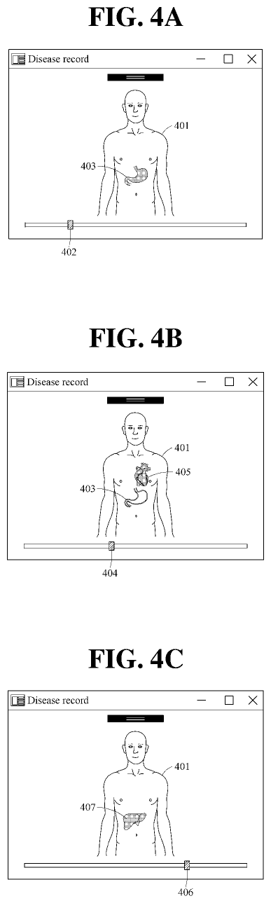

For example, as illustrated in FIGS. 4A and 4B, as a time to be selected by an operation of the GUI controller changes from a first point 402 to a second point 404 in a direction receding from a reference point, the identifier 110 may verify a time (e.g., December 1, 2017) corresponding to the second point 404, and identify a first disease (e.g., heart disease) occurring at the verified time (December 1, 2017) and a second disease (e.g., stomach disorder) occurring prior to the time (December 1, 2017) and not completely cured yet. When the time selected by the operation of the GUI controller is at the second point 404 as illustrated in FIG. 4B, the processor 120 may visualize an image 405 of the first disease (heart disease) in a body image 401, and indicate the occurrence of the first disease (heart disease). In addition, when the time selected by the operation of the GUI controller is at the first point 402 as illustrated in FIG. 4A, the processor 120 may visualize an image 403 of the second disease (stomach disorder) by changing an opaque color of the image 403 as illustrated in FIG. 4A to a transparent color as illustrated in FIG. 4B, and indicate that a state of the second disease (stomach disorder) is improving through a medical treatment. As described above, the doctor, or a medical practitioner, may verify only at a single glance occurrence and progression of a disease, a treatment progress of the disease and a full recovery from the disease, and the like based on a lapse of time, through an image simply by an operation of the GUI controller.

Referring to FIG. 1, the processor 120 includes a time changer 124 and a direction changer 125.

The time changer 124 may change a time to be displayed on the patient-condition-check-display based on a movement of a first GUI controller, for example, a scroll bar.

That is, as a time to be selected by an operation of the first GUI controller changes in a direction receding from a reference point, the time changer 124 may change the time to be displayed on the patient-condition-check-display, for example, from a previous time at which a disease occurs initially to a current time at which the patient-condition-check-display is requested.

Thus, a visualizer 121 to be described hereinafter may visualize, in the body image, an image representing a disease in chronological order.

Alternatively, when a play button on the patient-condition-check-display is selected or pressed, the processor 120 may store or display, as moving images (or video or movie) to be visualized in the body image, images of diseases occurring from an initial occurrence time of a disease up to a current time in chronological order.

For example, the processor 120 may store, as one or more still images or moving images (or movie or video), an image of a disease corresponding to an occurrence time of the disease. In this example, in response to a request for the patient-condition-check-display, the processor 120 may then configure the patient-condition-check-display by sequentially displaying the stored images on the patient-condition-check-display.

According to an example, the processor 120 may store, as one or more still images or moving images (or movie or video), an entirety or a portion of the patient-condition-check-display including the disease image, and sequentially display the stored still images or moving images (or movie or video) when a play button is selected or pressed.

Here, each time a fast-play button on the patient-condition-check-display is selected or pressed, the processor 120 may visualize, in the body image, the disease images while changing the disease images at a high speed by increasing a speed for the changing by two times, three times, and n times.

In addition, each time a back button on the patient-condition-check-display is selected or pressed, the processor 120 may visualize, in the body image, an image of a previously occurring disease while skipping to a set previous time, for example, an immediately previous occurrence time of a disease.

In addition, the processor 120 may perform the functions described above by recognizing a gesture such as, for example, a tapping gesture of a finger tapping on the patient-condition-check-display, or recognizing a change in the number of fingers touching the patient-condition-check-display. In addition, the processor 120 may perform the functions described above by recognizing a voice command such as, for example, “play,” “double speed,” and “back.”

Thus, a doctor or a medical practitioner may change and search for a disease image to be visualized in the body image on the patient-condition-check-display without an operation of a GUI controller or a button.

The direction changer 125 may change a direction in which the body image is to be displayed on the patient-condition-check-display to display, for example, an upper body, a lower body, a front side body, or a back side body, based on an operation of the second GUI controller, for example, a setting button or a shortcut key included in the patient-condition-check-display.

Referring to FIG. 1, the processor 120 also includes the visualizer 121, a changer 122, and a remover 123.

The visualizer 121 may visualize, in the body image, a disease image representing a body tissue, for example, an organ and a bone, in which a first disease occurs or a location at which the disease occurs.

For example, the identifier 110 may extract, from the DB 130, medical information generated at a time selected from the patient-condition-check-display by an operation of the first GUI controller, and the visualizer 121 may visualize, in the body image, an image of a disease identified from the medical information.

Here, when a full recovery time at which the disease is completely cured is not identified from the medical record of the patient, the visualizer 121 may visualize the disease image blinking to indicate that the disease is under treatment.

A full recovery time used herein may indicate a point in time at which a disease is improved and thus a treatment plan for the disease in a medical record of a patient who experiences the disease is changed from “under treatment” to “under observation,” or a point in time at which the disease under treatment is removed from the medical record. Alternatively, the full recovery time may indicate a point in time at which a name of the disease is mentioned as a past disease in the medical record or the disease is not verified from the medical record.

That is, when the disease continues being recorded or registered in a list of diseases in the EMR display, or a continued visit to a medical center for the disease is recorded, the visualizer 121 may verify that the disease is still under treatment without being completely cured yet, and visualize the disease image blinking.

In addition, when visualizing an image associated with the first disease, the visualizer 121 may visualize the image in a color different from that of an image associated with the second disease.

In addition, when the first disease and the second disease occur at a same region, the visualizer 121 may display an overlapping region of the first and second images using a hatched line such that the overlapping region is distinguishable. In addition, the visualizer 121 may change or adjust at least one of a color, a level of brightness, or a level of transparency of the overlapping region, or visualize a certain pattern in the region such that the overlapping region is distinguishable.

The changer 122 may change a color, a level of brightness, and a level of transparency of the disease image associated with the second disease that is visualized in the body image based on a treatment progress of the second disease and a level of severity of the second disease.

That is, through such a change in an image of a disease to be visualized in the body image, the changer 122 may visualize stepwise a level of severity of the disease and a treatment progress of the disease.

In addition, when an ROI in the body image is set on the patient-condition-check-display and a certain disease is identified from a body tissue included in the set ROI, the changer 122 may change a level of brightness and a size of a disease image to be visualized in the body image according to an operation of the first GUI controller based on medical information associated with the disease, and thus visualize progression or a state change of the disease. For example, the changer 122 may visualize a treatment progress of the disease through medication or drug administration for the disease by adjusting a color, a level of brightness, and a level of transparency of the disease image.

According to an example, the changer 122 may change at least one of a color, a level of brightness, or a level of transparency of a disease image to be visualized in the body image, based on a function with input parameters, which are, at least one of a frequency of visits to a hospital, a frequency of medical treatments the patient receives, or a blood test result.

The remover 123 may remove a disease image visualized in the body image from the patient-condition-check-display, when a time selected by an operation of the first GUI controller passes through a point corresponding to a full recovery time of a corresponding disease.

According to an example embodiment, simply by an operation of a first GUI controller, it is possible to verify at a single glance occurrence and progression of a disease, and a medical treatment progress of the disease and a full recovery from the disease, using images provided with a lapse of time.

According to another example embodiment, the patient-condition-check-display providing system 100 may include the identifier 110 configured to set, as an ROI, a region dragged or clicked in a body image of the patient-condition-check-display, and identify a disease image representing a disease occurring in a body tissue located in the ROI from a medical record of a patient; and the processor 120 configured to visualize a disease image representing the identified disease in the body image based on an occurrence time of the disease and provide the patient-condition-check-display including the disease image visualized in the body image. When a time to be displayed on the patient-condition-check-display changes by an operation of the first GUI controller, the processor 120 may change a color, a level of brightness, and a level of transparency of the disease image based on medical information including data of a medical treatment progress of the disease, and thus visualize progression or a state change of the disease in relation to the treatment progress of the disease.

FIG. 2 is a diagram illustrating an example of how a patient-condition-check-display providing system outputs a prescription registration display to a terminal of a doctor in connection with a patient-condition-check-display according to an example embodiment.

Referring to FIG. 2, when an operating time is not explicitly set or at a certain point in time, a patient-condition-check-display providing system may search for a medical record of a patient from the EMR system 101 of FIG. 1, and identify diseases previously occurring in the patient.

In addition, when patient information is registered or when a request for outpatient care is received, or at a point in time set every day, the patient-condition-check-display providing system may search for a medical record of a corresponding patient from the EMR system 101 and identify diseases previously occurring in the patient.

In addition, when an item 211 associated with registering a prescription drug is selected or pressed from an EMR display 210 output to a terminal of a doctor who examines a patient, the patient-condition-check-display providing system may search for a medical record of the patient from the EMR system 101 and identify a disease previously occurring in the patient, for example, a stomach disorder.

The patient-condition-check-display providing system may then provide, onto the EMR display 210, a patient-condition-check-display 220 including a body image 221 and a GUI controller 222, and visualize a disease image (e.g., stomach shape) representing the identified disease (e.g., stomach disorder) in a region (e.g., center in an upper body) of the body image 221 in which the disease occurs. The patient-condition-check-display providing system may reflect a status of a medical treatment for the disease according to an operation of the GUI controller 222, and change at least one of a color, a level of brightness, a level of transparency, or a size of the disease image to be visualized.

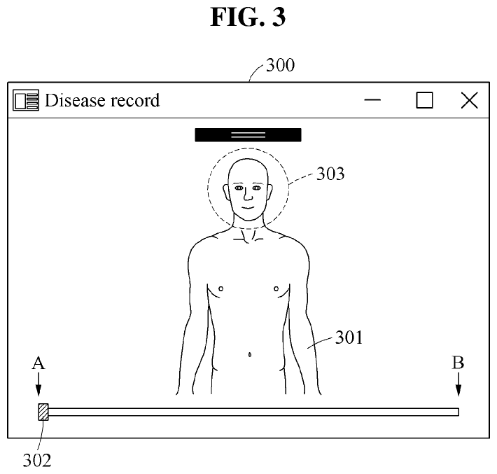

FIG. 3 is a diagram illustrating an example of a patient-condition-check-display provided by a patient-condition-check-display providing system according to an example embodiment.

Referring to FIG. 3, in response to a request for searching an EMR display for a medical history of a patient, a patient-condition-check-display providing system may provide or display a patient-condition-check-display 300 including a body image 301 and a GUI controller 302, for example, a scroll bar, on the EMR display, or as a portion of the EMR display or in replacement of the EMR display.

The GUI controller 302 may change a time to be displayed on the patient-condition-check-display 300. That is, the GUI controller 302 may be used to set a time to be displayed on the patient-condition-check-display 300 in relation to a disease for which the patient is receiving or has received a medical treatment.

As illustrated in FIG. 3, when a time selected by an operation of the GUI controller 302 is located at a reference point A, the patient-condition-check-display providing system may visualize a normal state or condition in which the patient has no disease.

Afterwards, as a time selected by an operation of the GUI controller 302 moves or changes in a direction receding from the reference point A, the patient-condition-check-display providing system may identify whether there is a disease from a medical record or medical information of the patient corresponding to a time corresponding to a point to which the GUI controller 302 moves. When there is a disease, the patient-condition-check-display providing system may visualize a disease image representing the disease in the body image 301 to display occurrence of the disease.

Afterwards, as a time selected by an operation of the GUI controller 302 moves or changes in a direction approaching a current point B, the patient-condition-check-display providing system may identify data of a treatment progress of the disease (e.g., data of drug administration, surgery, hospitalization, blood test result abnormality, frequency of receiving medical treatments) from the medical record of the patient, and change a color, a level of brightness, a level of transparency, and a size of the disease image. Thus, the patient-condition-check-display providing system may visualize, in a form of an image, a whole process of progression involving, for example, occurrence of the disease, and a medical treatment for the disease and a full recovery from the disease.

According to an example, the patient-condition-check-display providing system may search for a disease image by setting an ROI 303 in the body image 301 of the patient-condition-check-display 300. In detail, the patient-condition-check-display providing system may search a medical record for a disease (e.g., brain disease) occurring in a body tissue (e.g., brain, face, eye, etc.) included in the ROI 303 set by a user, and visualize a disease image representing the retrieved disease based on a movement of the GUI controller 302, for example, a scroll bar.

For example, the patient-condition-check-display providing system may set, as the ROI 303, a region in the body image 301 dragged by the user or a region in the body image 301 clicked by the user, and search for the disease image of the disease occurring in the ROI 303 and then visualize the retrieved disease image in the body image 301. For another example, the patient-condition-check-display 300 or the EMR display may include a search window to retrieve a disease. In such example, when a user inputs, to the search window, a text of a disease name, a disease occurrence location, and a disease occurrence time, the patient-condition-check-display providing system may search for a disease matching the input text from the medical record of the patient and visualize a disease image representing the retrieved image in the body image 301.

The patient-condition-check-display providing system may combine the ROI 303 and the text input to the search window based on a selected search condition, for example, “AND,” “OR,” and “NOT,” and search for a disease satisfying a result of the combining from the medical record of the patient.

For example, when the ROI 303 is set by a user and a text, for example, “tumor,” is input to the search window, the patient-condition-check-display providing system may search for a tumor occurring in the ROI 303 from the medical record of the patient in response to “AND” being selected as the search condition. In response to “OR” being selected as the search condition, the patient-condition-check-display providing system may search for all diseases and tumors occurring in the ROI 303 from the medical record of the patient. In response to “NOT” being selected as the search condition, the patient-condition-check-display providing system may search for all diseases occurring in the ROI 303, excluding a tumor.

As described above, the patient-condition-check-display providing system may provide a function of retrieving a disease image in various ways on the patient-condition-check-display 300.

FIGS. 4A through 4C are diagrams illustrating an example of visualizing occurrence and progression of a disease on a patient-condition-check-display provided by a patient-condition-check-display providing system according to an example embodiment.

FIG. 4A illustrates a stomach disorder initially occurring in a patient. FIG. 4B illustrates a heart disease occurring while the stomach disorder is under treatment at a time to which a GUI controller moves. FIG. 4C illustrates hepatitis newly occurring when both the stomach disorder and the heart disease are cured as a GUI controller moves.

Referring to FIG. 4A, as a time selected by an operation of a GUI controller moves or changes rightwards receding from a reference point and reaches a first point 402, a patient-condition-check-display providing system may visualize a stomach shape image 403 in a body image 401, and provide a notification of occurrence of a stomach disorder. Referring to FIG. 4B, as a time selected by an operation of the GUI controller moves or changes to a second point 404, the patient-condition-check-display providing system may transparently visualize the stomach shape image 403 in the body image 401 to provide a notification that the stomach disorder is under treatment, and visualize a heart shape image 405 in the body image 401 to provide a notification of occurrence of a heart disease.

Referring to FIG. 4C, as a time selected by an operation of the GUI controller moves or changes to a third point 406, the patient-condition-check-display providing system may remove the stomach shape image 403 and the heart shape image 405 that are previously visualized in the body image 401 to provide a notification that the stomach disorder and the heart disease are completely cured, and visualize a liver shape image 407 in the body image 401 to provide a notification of occurrence of hepatitis.

As described above, the patient-condition-check-display providing system may support a function of allowing a doctor or a medical practitioner to verify, all at once, occurrence and progression of a disease, and a treatment progress of the disease and a full recovery from the disease, using images to be shown according to a lapse of time, simply by operating a GUI controller.

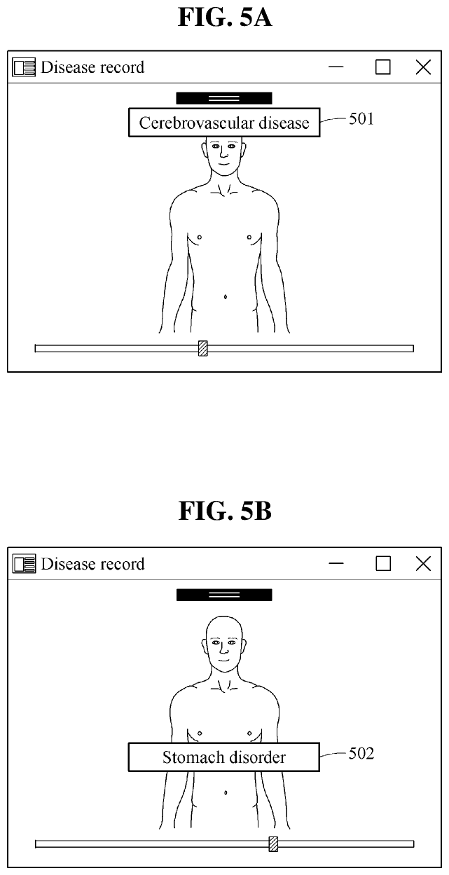

FIGS. 5A and 5B are diagrams illustrating another example of visualizing occurrence and progression of a disease on a patient-condition-check-display provided by a patient-condition-check-display providing system according to an example embodiment.

Referring to FIGS. 5A and 5B, a patient-condition-check-display providing system may display a speech bubble by associating a disease with a location in a body image at which the disease occurs, and visualize, in a text form, information associated with a name of the disease, an occurrence time of the disease, a level of severity of the disease, and a current state of the disease through the speech bubble.

In detail, as illustrated in FIG. 5A, as a GUI controller moves rightwards from a reference point, the patient-condition-check-display providing system may display a speech bubble 501 to indicate a location in a body image, for example, a brain, at which a disease occurs, and visualize, in the speech bubble 501, a cerebrovascular disease which is a name of the disease. Through this, the patient-condition-check-display providing system may provide a medical practitioner or a doctor with sketchy information about occurrence and an occurrence location of the cerebrovascular disease.

In addition, as illustrated in FIG. 5B, as the GUI controller further moves rightwards, the patient-condition-check-display providing system may display a speech bubble 502, rather than canceling the speech bubble 501, to visualize a stomach disorder which is a name of a corresponding disease through the speech bubble 502. Through this, the patient-condition-check-display providing system may provide a medical practitioner or a doctor with information about a full recovery from the cerebrovascular disease and new information about occurrence of the stomach disorder.

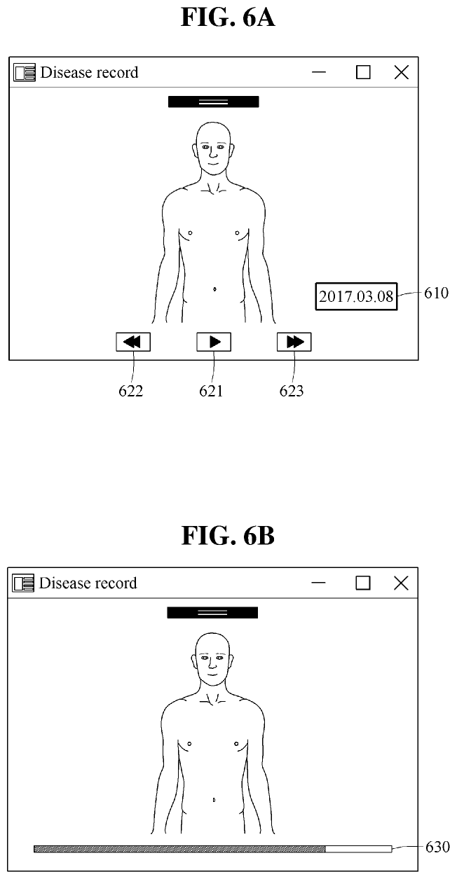

FIGS. 6A and 6B are diagrams illustrating an example of searching for an disease image representing a disease from a patient-condition-check-display provided by a patient-condition-check-display providing system according to an example embodiment.

Referring to FIG. 6A, a patient-condition-check-display providing system may provide a patient-condition-check-display further including a play button 621, a back button 622, and a fast button 623 to facilitate a search for a disease image by an operation of such buttons.

The patient-condition-check-display providing system may display, in a text form, on the patient-condition-check-display, a time (e.g., March 8, 2017) 610 to be visualized by an operation of each of the buttons 621 through 623.

When the play button 621 is clicked, the patient-condition-check-display providing system may generate and display a previously generated video (or moving image) or a video that chronologically visualizes, in a body image, disease images from an initial time of occurrence of a disease up to a current time.

Alternatively, the patient-condition-check-display providing system may store, as one or more still images or moving images (or movie or video), an entirety or a portion of the patient-condition-check-display including a disease image. When the play button 621 is selected or pressed, the patient-condition-check-display providing system may sequentially display the stored still images, or moving images or a movie.

Here, the patient-condition-check-display providing system may further output a progress bar 630 as illustrated in FIG. 6B to provide information about how far the moving image or video proceeds and about a time (e.g., March 8, 2017) at which the visualization is embodied on the patient-condition-check-display.

In addition, each time the back button 622 is clicked, the patient-condition-check-display providing system may change the time 610 visualized on the patient-condition-check-display to a previous time, for example, an occurrence time of an initial disease, such that a previous disease image is retrieved in reverse order.

For example, each time the back button 622 is clicked, the patient-condition-check-display providing system may skip to a preset previous time, for example, a week earlier, and visualize an image of a previously occurring disease in a body image.

In addition, each time the fast button 623 is clicked, the patient-condition-check-display providing system may change the time 610 visualized on the patient-condition-check-display to approach a current time such that a more recent image of a disease is retrieved.

For example, each time the fast button 623 is clicked, the patient-condition-check-display providing system may increase a play speed of a movie or video by two times, three times, and n times, such that a recent image of a disease is retrieved faster.

As described above, the patient-condition-check-display providing system may provide a function of retrieving a disease image in various ways from the patient-condition-check-display, and thus allow a medical practitioner to simply search for a disease image at a time at which the medical practitioner desires to verify a disease, without operating a scroll bar.

Hereinafter, how a patient-condition-check-display providing system operates will be described in detail.

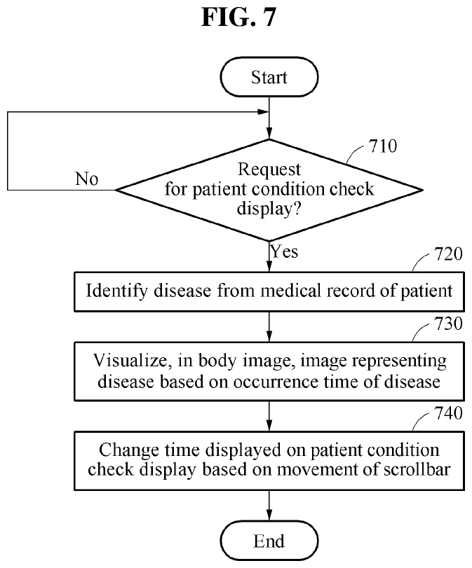

FIG. 7 is a flowchart illustrating an example of a method of providing a patient-condition-check-display according to an example embodiment.

The method of providing a patient-condition-check-display may be performed by a patient-condition-check-display providing system, for example, the patient-condition-check-display providing system 100 of FIG. 1.

Referring to FIG. 7, in operation 710, the patient-condition-check-display providing system 100 verifies whether there is a request for a patient-condition-check-display from a terminal of a doctor who treats or examines a patient.

For example, when an item associated with checking a medical history or an item associated with registering a prescription drug is selected from a patient EMR display, a treatment such as hospitalization, surgery, medical examination, and the like is input, or the patient visits a hospital or a medical center for the first time and thus information associated with a medical history of the patient is needed, the patient-condition-check-display providing system 100 may recognize the request for the patient-condition-check-display.

In operation 720, when the request for the patient-condition-check-display is verified, the patient-condition-check-display providing system 100 identifies a disease occurring in the patient from a medical record of the patient.

For example, the patient-condition-check-display providing system 100 may search an EMR system for a medical record issued by all medical centers including other medical centers visited by the patient, and identify all diseases or disorders previously occurring in the patient based on a current time at which the request is made.

In addition, when identifying the disease from the medical record of the patient, the patient-condition-check-display providing system 100 may further identify data of a treatment progress including, for example, an occurrence time of the disease, a full recovery time at which the disease is completely cured, a portion or region in which the disease occurs, a size and a shape of the disease, a medicine or drug administered to the patient for the disease, a surgery or surgical operation the patient underwent for the disease, a hospitalization history, a severity of the disease, a frequency of visits to the hospital or a frequency of medical treatments the patient received, and the like.

In operation 730, the patient-condition-check-display providing system 100 provides the patient-condition-check-display by visualizing, in a body image, a disease image representing the disease based on an occurrence time of the disease.

For example, referring back to FIG. 2, the patient-condition-check-display providing system 100 may search the EMR system 101 for a medical record of a patient when a request for outpatient care is received, and identify information needed for the doctor to know before the doctor provides a medical treatment to the patient. The information may include, for example, information about another disease or disorder, for example, a stomach disorder for which the patient receives a medical treatment from another doctor. The patient-condition-check-display providing system 100 may then provide, onto the EMR display 210, the patient-condition-check-display 220 including the body image 221 and the GUI controller 222, and visualize a stomach shape image representing the stomach disorder in a region of a body of the patient in the body image 221, for example, a center in an upper body of the patient, at which the stomach disorder occurs.

In operation 740, the patient-condition-check-display providing system 100 changes a time to be displayed on the patient-condition-check-display based on a movement of a GUI controller.

For example, referring back to FIG. 2, the patient-condition-check-display providing system 100 may adjust a size and a level of transparency of the stomach shape image to be visualized in the body image 221 based on a movement of the GUI controller 222 included in the patient-condition-check-display 220, and visualize a treatment status of the stomach disorder.

As described above, the patient-condition-check-display providing system 100 may visualize, in a body image, images representing diseases identified from a medical record of a patient in sequential order based on respective occurrence times of the diseases, and thus facilitate identification of occurrence of the diseases at each point in time and a change or progression of the diseases based on a lapse of time.

According to example embodiments, there is provided a patient-condition-check-display providing system and method. The system and method may allow a medical practitioner to rapidly and intuitively identify a medical history of a patient and a current state of a disease the patient suffers, through an image, and thus allow the medical practitioner to provide a desirable medical treatment, prescription, and diagnosis to the patient within a relatively short period of time.

Through an image that allows a medical practitioner to effectively identify, all at once, various diseases previously occurring in a patient based on a lapse of time, the system and method may also allow the medical practitioner to identify a relationship between diseases including, for example, an initial disease, and a complication or a secondary disease or a side effect that occurs during a medical treatment, and thus allow the medical practitioner to provide a correct medical treatment and a safe prescription to the patient.

In addition, the system and method may provide, onto an EMR display run on a terminal of a medical practitioner, a patient-condition-check-display that visualizes, in a body image, an image representing a disease occurring in a patient based on a lapse of time, and thus allow the medical practitioner to readily identify a medical history and a current condition of the patient through the image in lieu of a complex form of text.

In addition, the system and method may facilitate a retrieval of or a search for progression of a disease at a certain point in time based on an operation of a GUI controller.

Simply by an operation of the GUI controller, the system and method may allow a medical practitioner to verify, all at once, occurrence and progression of a disease and a treatment progress of the disease, through an image provided based on a lapse of time.

In addition, the system and method may accumulate summaries of previous diseases a patient suffers in chronological order, record the accumulated summaries as medical information of the patient, and allow a medical practitioner to retrieve a summary he/she desires to refer to through a scrolling operation.

In addition, the system and method may provide a human body map obtained by visualizing a disease image in a body image based on a lapse of time, and allow a medical practitioner to conveniently monitor progression of a disease through the human body map simply using a GUI controller.

Further, the system and method may store or record information about progression of a disease of a patient in a form of an image, for example, a human body map, and support a function of retrieving progression or a state of a disease at a certain point in time simply using a GUI controller.

The units described herein may be implemented using hardware components and software components. For example, the hardware components may include microphones, amplifiers, band-pass filters, audio to digital convertors, non-transitory computer memory and processing devices. A processing device may be implemented using one or more general-purpose or special purpose computers, such as, for example, a processor, a controller and an arithmetic logic unit (ALU), a digital signal processor, a microcomputer, a field programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such a parallel processors.

The software may include a computer program, a piece of code, an instruction, or some combination thereof, to independently or collectively instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. The software and data may be stored by one or more non-transitory computer readable recording mediums. The non-transitory computer readable recording medium may include any data storage device that can store data which can be thereafter read by a computer system or processing device.

The methods according to the above-described example embodiments may be recorded in non-transitory computer-readable media including program instructions to implement various operations of the above-described example embodiments. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher level code that may be executed by the computer using an interpreter. The above-described devices may be configured to act as one or more software modules in order to perform the operations of the above-described example embodiments, or vice versa.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

What is claimed is:

1. A method of providing a patient-condition-check-display, comprising:
identifying a disease occurring in a patient from a medical record of the patient; and
visualizing, in a body image, a disease image representing the disease based on an occurrence time of the disease, and providing the patient-condition-check-display including the disease image visualized in the body image.

2. The method of claim 1, further comprising:
changing a time to be displayed on the patient-condition-check-display based on an operation of a first graphical user interface (GUI) controller; and
changing a size or a direction of the body image to be displayed on the patient-condition-check-display based on an operation of a second GUI controller different from the first GUI controller.

3. The method of claim 2, wherein the identifying of the disease comprises:
verifying a time selected by an operation of the first GUI controller; and
identifying a first disease occurring at the verified time, and a second disease occurring prior to the time and not completely cured yet.

4. The method of claim 3, wherein the providing of the patient-condition-check-display comprises:
visualizing, in the body image, a disease image representing a body tissue at which the first disease occurs; and
changing a level of brightness of a disease image associated with the second disease and visualized in the body image, based on a treatment progress of the second disease and a level of severity of the second disease.

5. The method of claim 3, wherein the providing of the patient-condition-check-display comprises:
visualizing a disease image associated with the first disease in a color different from a color for a disease image associated with the second disease; and
when the first disease occurs in a same region as the second disease, displaying an overlapping region of the disease images using a hatched line or a pattern, or changing at least one of a color, a level of brightness, or a level of transparency of the overlapping region.

6. The method of claim 2, when the first GUI controller passes through a point corresponding to a full recovery time of the disease to be identified from the medical record of the patient, further comprising:
removing the disease image visualized in the body image from the patient-condition-check-display.

7. The method of claim 1, wherein, when a full recovery time of the disease is not identified from the medical record of the patient, the providing of the patient-condition-check-display comprises:
visualizing, in the body image, the disease image blinking.

8. The method of claim 1, wherein, when a region of interest (ROI) is set in the body image on the patient-condition-check-display, and a disease is identified from a body tissue included in the ROI, further comprising:
visualizing progression of the disease by changing a level of brightness and a size of a disease image to be visualized in the body image according to an operation of a first GUI controller based on medical treatment information associated with the disease.

9. The method of claim 1, further comprising:
storing, in a database (DB), a portion or an entirety of the patient-condition-check-display including the disease image as one or more still images, or moving images or a movie based on the occurrence time of the disease,
wherein the providing of the patient-condition-check-display comprises:
providing the patient-condition-check-display by sequentially displaying the one or more still images, or moving images or movie when a play button is selected.

10. The method of claim 1, wherein, when a GUI event occurs in the body image, the providing of the patient-condition-check-display comprises:
providing the patient-condition-check-display by displaying a speech bubble in association with a region in which the GUI event occurs, and by visualizing, in a text form, information associated with at least one of a name of a disease occurring in the region, an occurrence time of the disease, a level of severity of the disease, or a current state of the disease.

11. The method of claim 1, further comprising:
changing at least one of a color, a level of brightness, or a level of transparency of the disease image to be visualized in the body image, based on a function having, as a factor, at least one of a frequency of visits of the patient to a hospital or a blood test result of the patient.

12. The method of claim 1, further comprising:
when a region in the body image is dragged or clicked, selecting the region as an ROI and searching for a disease occurring in a body tissue located in the ROI from the medical record of the patient; or
when at least one of a disease name, a disease occurrence location, or a disease occurrence time is input as a text to a search box on the patient-condition-check-display, searching a disease associated with the input text from the medical record of the patient; and
visualizing, in the body image, a disease image representing the disease retrieved by the searching.

13. The method of claim 12, further comprising:
combining the ROI and the text input to the search box, and searching for a disease satisfying a result of the combining above conditions from the medical record of the patient.

14. The method of claim 1, when a GUI event occurs in the body image, further comprising:
displaying a context menu including at least one of items associated with the disease including a detailed information display item, a prescription item, a progression search item, and a progression note item; and
changing a medical treatment display operating in a terminal of a doctor based on an item selected from the displayed context menu.

15. The method of claim 14, wherein the changing of the medical treatment display comprises:
changing the medical treatment display based on a time to be displayed on the patient-condition-check-display.

16. The method of claim 1, wherein the identifying of the disease further comprises:
identifying the disease by selecting, from the medical record of the patient, at least one of a specific disease, diseases associated with each other, a disease occurring in a region selected as an ROI, or a disease occurring at a specific time.

17. The method of claim 1, wherein the providing of the patient-condition-check-display comprises:
sequentially visualizing, in the body image, a plurality of disease images associated with diseases occurring as a time selected by an operation of a GUI controller moves or changes rightwards from a reference point; and
during the visualizing, removing a notification of “completely cured” in association with a first disease image of a first disease that is completely cured among the disease images after displaying the notification, and transparently displaying a notification of “under treatment” in association with a second disease image of a second disease that is not completely cured yet among the disease images.

18. A system for providing a patient-condition-check-display, comprising:
an identifier configured to identify a disease occurring in a patient from a medical record of the patient; and
a processor configured to visualize, in a body image, a disease image representing the disease based on an occurrence time of the disease, and provide the patient-condition-check-display including the disease image visualized in the body image.

19. The system of claim 18, wherein the processor is configured to:
change a time to be displayed on the patient-condition-check-display based on an operation of a first graphical user interface (GUI) controller; and
change a size or a direction of the body image to be displayed on the patient-condition-check-display based on an operation of a second GUI controller different from the first GUI controller.

20. The system of claim 18, wherein the identifier is configured to:
verify a time selected by an operation of a first GUI controller;
identify a first disease occurring at the verified time and a second disease occurring prior to the time and not completely cured yet, and
the processor is configured to:
visualize, in the body image, a disease image representing a body tissue at which the first disease occurs; and
change a level of brightness of a disease image associated with the second disease and visualized in the body image based on a treatment progress of the second disease and a level of severity of the second disease.
